(19) United States
(12) Patent Application Publication
(10) Pub. No.: US 2015/0320926 A1
(43) Pub. Date: Nov. 12, 2015

(54) VENTED CAP FOR CANNULA USED IN PERFUSION CIRCUIT

(71) Applicant: TERUMO CARDIOVASCULAR SYSTEMS CORPORATION, ANN ARBOR, MI (US)

(72) Inventors: Douglas M. Fitzpatrick, Huron Township, MI (US); Kevin G. Obrigkeit, Plymouth, MI (US)

(73) Assignee: TERUMO CARDIOVASCULAR SYSTEMS CORPORATION, ANN ARBOR, MI (US)

(21) Appl. No.: 14/176,628

(22) Filed: Feb. 10, 2014

(51) Int. Cl. A61M 1/36; A61M 39/20

(52) U.S. Cl. CPC A61M 1/3659; A61M 1/3666; A61M 39/20; A61M 2039/205

(57) ABSTRACT

A removable cap for a cardiovascular cannula has a sleeve section with an inner surface adapted to receive a connector end of the cannula. The cap has a top section closing a closed end of the sleeve section. A pull tab extends radially from an open end of the sleeve section. A pair of tear seams are sunk into the sleeve section and the top section defining a tear strip in the cap. The sleeve section includes a segmented snap ring protruding radially inward on the inner surface of the sleeve section except at a pair of gaps coinciding with the pair of tear seams. The top section includes a seal cylinder projecting concentrically from the top section to seal against an inside surface of the cannula connector end.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to a perfusion cannula for insertion into the cardiovascular system, and, more specifically, to a vented cap retained on a proximal end of a cannula that is easily removed for connection into a perfusion circuit after priming.

During cardiac bypass surgery, a patient's blood is redirected through an extracorporeal perfusion circuit typically including various items such as a venous cannula, PVC tubing, a reservoir, a pump, an oxygenator, an arterial filter, and a return cannula. The most common type of return cannula is the arterial (e.g., aortic) cannula with a tapered plastic tip at the distal end for insertion through an incision in the aorta.

A connector or adapter at the proximal end is initially covered by a sealed cap to prevent significant blood loss once the tip is inserted into the aorta. Vent holes in the cap (and/or a manually-controlled luer lock vent adjacent the cap) may be provided to remove air from the cannula as it initially fills with blood (known as priming). After priming, the cap is removed by the surgeon, and then the connector/adapter is connected to tubing that provides oxygenated blood from the pump/oxygenator.

The proximal connector of the arterial cannula may be comprised of a barbed fitting. A typical cap is made of a flexible elastomer in the shape of a cylinder which is closed at one end and has an internal sealing ring that engages a barb on the cannula connector. A tear strip and tear tab are formed on the cap to facilitate removal of the cap when desired. The conventional tear strip is defined by seams or grooves extending from the open end and up a portion of the side of the cap. By pulling the tear tab, the tear strip splits partly away from the cap on one side so that a lower portion of the cap no longer grips the cannula.

In a conventional cap, the tear seams or grooves may extend across the internal sealing ring. Even when they extend that far, however, the resiliency of the upper portion of the cap tends to continue to hold it in place on the cannula because the sealing function of the cap dictates a sufficiently snug fit to avoid leakage prior to tearing of the cap. Thus, removal of prior art caps has required a second step in which the surgeon grasps the cap to pull the remaining portion off of the cannula. It would be desirable to provide for a one-step removal of the cap by merely pulling on the tear tab, while maintaining robust sealing characteristics before initiating removal of the cap.

### SUMMARY OF THE INVENTION

In one aspect of the invention, a removable cap is provided for a cardiovascular cannula. The cap has a sleeve section with an inner surface adapted to receive a connector end of the cannula. The cap has a top section closing a closed end of the sleeve section and extending substantially perpendicular to the sleeve section. The cap has a pull tab extending radially from an open end of the sleeve section and having first and second edges. A pair of tear seams are sunk into the sleeve section and the top section defining a tear strip in the cap. The tear seams include a sleeve portion extending substantially in parallel and substantially axially from the first and second edges of the pull tab to the top section. The tear seams have a top portion continuous with the sleeve portion and partially circumscribing the top section. The sleeve section includes a segmented snap ring protruding radially inward on the inner surface of the sleeve section except at a pair of gaps coinciding with the pair of tear seams. The top section includes a seal cylinder projecting concentrically from the top section to seal against an inside surface of the cannula connector end.

### BRIEF DESCRIPTION OF THE DRAWINGS

FIG. 1 is a plan view showing an aortic cannula.

FIG. 2 is a cross section of a prior art cap on a cannula connector.

FIG. 3 is a perspective view of the cap of FIG. 2.

FIG. 4 is a bottom view of the cap of FIG. 2.

FIG. 5 is a perspective view of one embodiment of a cap of the present invention.

FIG. 6 is a top view of the cap of FIG. 5.

FIG. 7 is a bottom view of the cap of FIG. 5.

FIG. 8 is a perspective and horizontal cross-sectional view of the cap of FIG. 5.

FIG. 9 is a vertical cross section of the cap of FIG. 5.

FIG. 10 is a vertical cross section of the cap of FIG. 5 installed on a proximal connector of an arterial cannula.

### DETAILED DESCRIPTION OF PREFERRED EMBODIMENTS

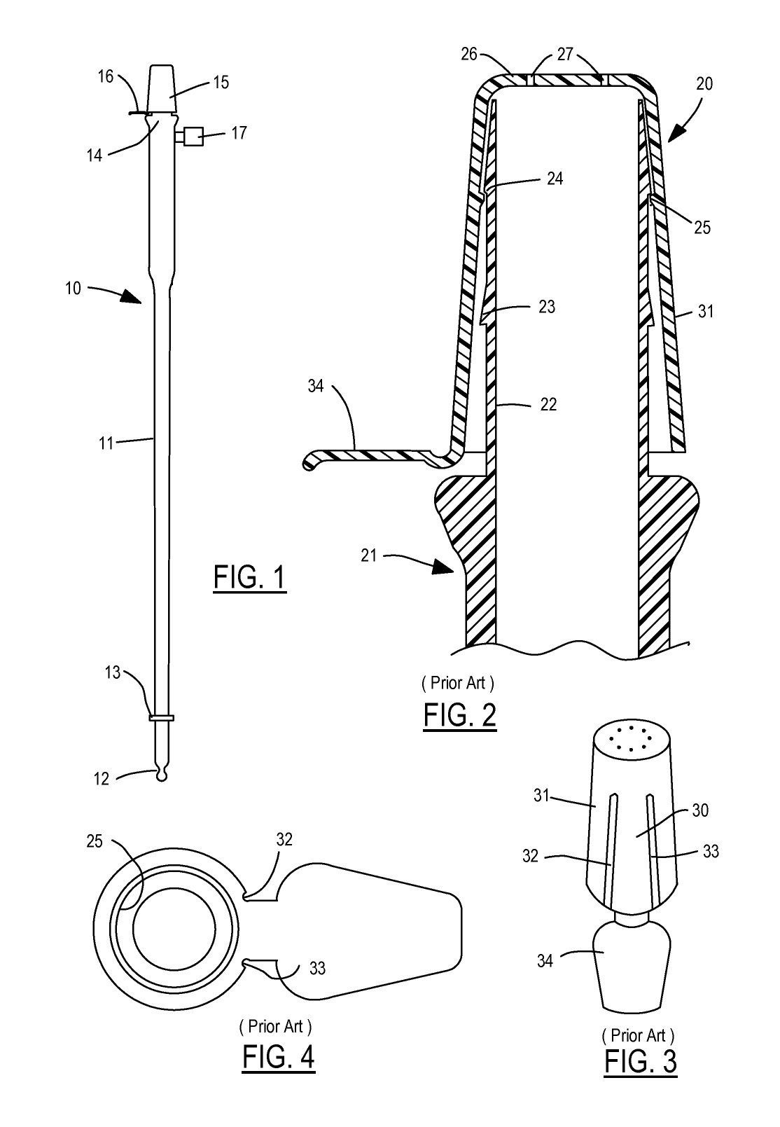

Referring now to FIG. 1, an arterial cannula 10 includes a lumen 11 having a tip 12 at the distal end adapted to be placed into the cardiovascular system of a patient. A suturing ring 13 may be provided near the tip 12 for attaching to a surface of the patient. The proximal end of lumen 11 has a connector 14 which is covered (prior to and during initial insertion into a patient) by a cap 15 including a pull tab 16 to facilitate removal. A Luer lock 17 may be provided for air removal as known in the art.

FIG. 2 shows one prior art embodiment of a cap 20 on a cannula connector end 21 in greater detail. Cap 20 is molded from a low density polyethylene (PE) and cannula end 21 is molded with another biocompatible plastic such as PTFE. Connector end 21 includes a cylindrical body 22 having external barbs 23 and 24 for creating a good seal when connected to the tubing of a perfusion circuit. Cap 20 includes a snap ring 25 on its internal surface for mating with barb 24 to provide both positive retention and an adequate seal that prevents blood loss during the priming of the cannula.

A top section 26 of cap 20 includes a plurality of small apertures 27 for venting air. Thus, when the distal tip is inserted into the cardiovascular system (e.g., into the aorta), blood enters the cannula lumen and flows toward connector end 21. As blood enters the cannula, air exits through apertures 27. Apertures 27 may taper to an exit diameter of about 0.004 inches so that they are large enough to vent air but are sufficiently small that little if any blood can leak through them once the lumen is fully primed.

When the surgeon is ready to attach other tubing to the primed cannula, the cap must be first be removed. Removal is achieved as shown in FIGS. 2-4 based on the use of a known tear strip 30 formed in a sleeve section 31 of cap 20 between a pair of score lines 32 and 33. Tear strip 30 is joined with a pull tab 34, allowing the surgeon to point upward upon pull tab 34 to separate tear strip 30 along score lines 32 and 33. Score lines 32 and 33 may extend longitudinally across the region of snap ring 25, resulting in an increased thickness to be torn through (an an increased effort) at the position of snap ring 25. With tear strip separated, cap 20 is partially released from connector 21. Final removal is often done by grasping the loosened part of sleeve section 31 (e.g., next to tear strip 30) and peeling off cap 20.

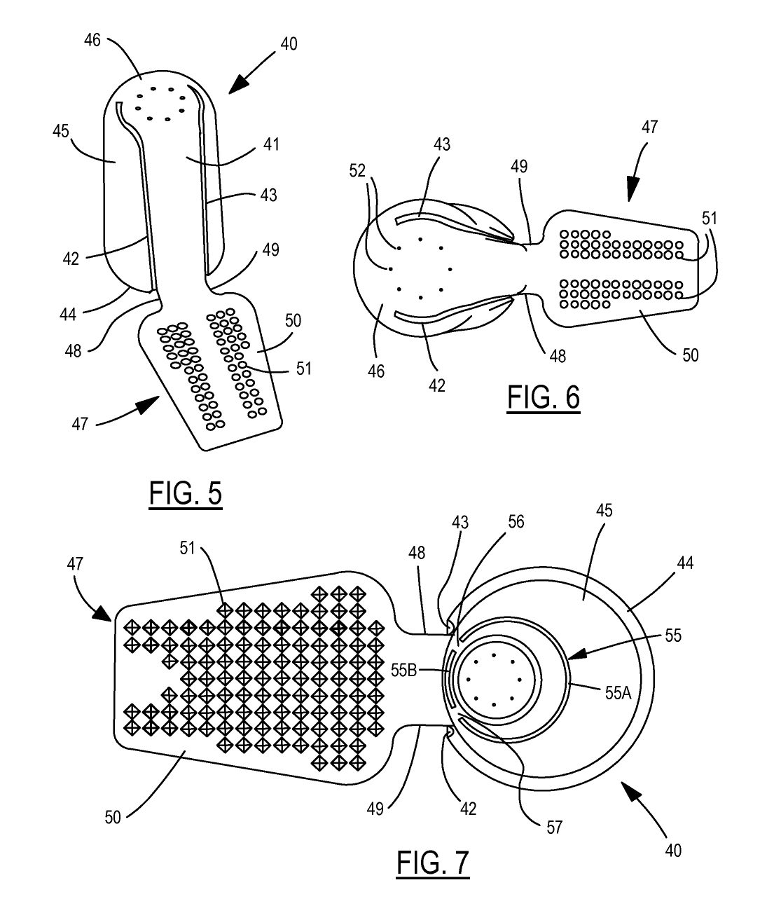

Embodiments of the present invention shown in FIGS. 5-10 employ various modified features to achieve easy cap removal in one smooth step while maintaining robust cap retention, effective sealing of liquid, and venting of gas during priming.

The perspective view of FIG. 5 shows a cap 40 with an improved tear strip 41 between tear seams 42 and 43. Seams 42 and 43 are sunk into the outer surface of cap 40 and extend from an open end 44 of cap 40 along a sleeve section 45 and continuing onto a top section 46 that closes an upper end of sleeve section 45. Top section 46 is substantially perpendicular to sleeve section 45. Tear seams 42 and 43 have a sleeve portion where they extend substantially in parallel and in an axial direction along sleeve section 45. Tear seams 42 and 43 also have a top portion where they turn to follow along an outer edge of top section 46, so that they partially circumscribe the top section.

A pull tab 47 extends radially from open end 44 of cap 40 and has first and second edges 48 and 49 that align with tear seams 42 and 43. Pull tab 47 preferably includes a grasping pad 50 that widens out from edges 48 and 49 to provide a size appropriate for grasping between the thumb and index finger of the user. A plurality of dimples 51 on the surface of pad 50 help establish a firm grasp for pulling tear strip 41 away from sleeve section 45. Since tear seams 42 and 43 extend all the way between the ends of sleeve section 45 and into top section 46, it is not necessary to peel the remaining portion of the sleeve section off of the cannula connector end.

As shown in FIGS. 5 and 6, top section 46 includes a plurality of apertures 52 for venting air from within the lumen of the cannula. Apertures 52 fall inside the circumscribed area between tear seams 43 and 42 and are aligned with the cannula lumen.

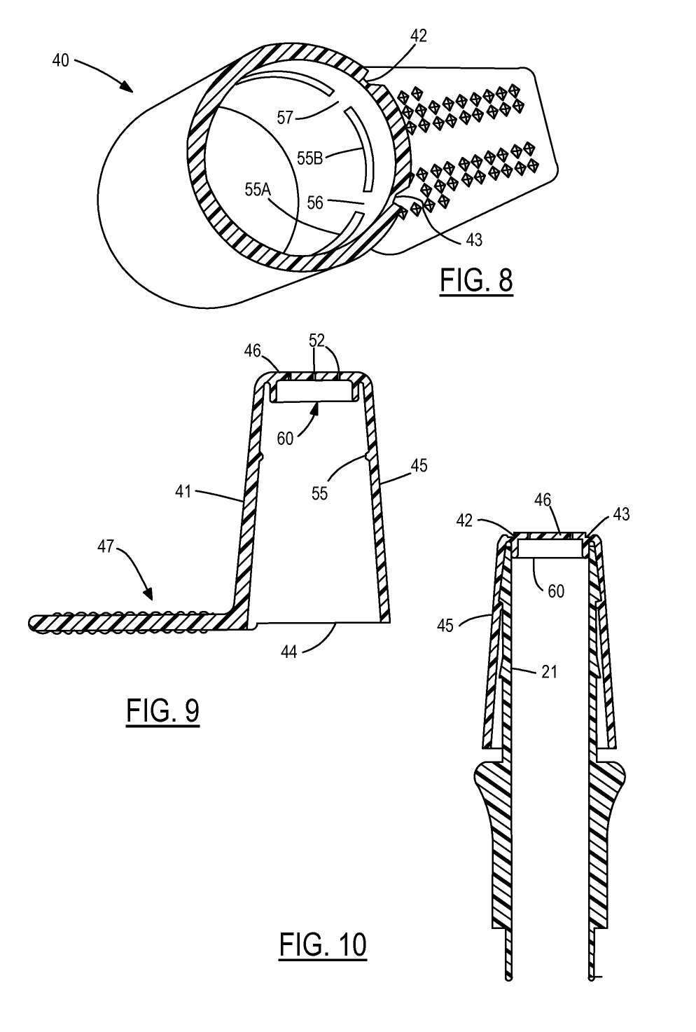

As shown in the bottom view of FIG. 7, cap 40 preferably includes a snap ring 55 protruding radially inward on the inner surface of sleeve section 45 for mating with a corresponding barb on the cannula connector end. Snap ring 55 is segmented into a first portion 55A and second portion 55B, resulting in a pair of gaps 56 and 57 coinciding with the edges of tear strip 41 between tear seams 42 and 43. Thus, the tearing action to remove the cap does not have to overcome an increased thickness at the location of the snap ring, thereby insuring a smooth, consistent tearing action across snap ring 55. In FIG. 8, an upper portion of cap 40 is cutaway to reveal snap ring 55 with gaps 57 and 56 corresponding to tear seams 42 and 43, respectively.

As a result of the segmentation of snap ring 55, a possibility of leakage of liquid past the snap ring may be increased. To compensate for such possibility, the present invention further adds a sealing cylinder 60 that projects concentrically from top section 46 as shown in FIG. 9. Seal cylinder 60 is dimensioned to seal against an inside surface of the cannula connector end as shown in FIG. 10. Thus, the end of cannula connector 21 is captured in a space between sleeve section 45 and cylinder 60 (at least until the tear strip is torn away). As shown in FIG. 10, tear seams 42 and 43 on top section 46 also partially circumscribe the area from which seal cylinder 60 projects. Consequently, during tearing of tear seams 42 and 43, cylinder 60 becomes partially removed from connector end 21 for easy removal of cap 40. Seal cylinder 60 is shown as a hollow cylindrical rib that engages the cannula connector end. However, other shapes such as a solid (i.e., filled) cylinder or rod could also be used.

Cap 40 is preferably formed as a single integrated unit. It is preferably comprised of polyethylene, which can be shaped using injection molding. The tear seams preferably have a U-shaped profile instead of the conventional V-shape, which results in a smoother tearing with a better controlled tearing force.

What is claimed is:

1. A removable cap for a cardiovascular cannula, comprising:
a sleeve section with an inner surface adapted to receive a connector end of the cannula;
a top section closing a closed end of the sleeve section and extending substantially perpendicular to the sleeve section; and
a pull tab extending radially from an open end of the sleeve section and having first and second edges;
wherein a pair of tear seams are sunk into the sleeve section and the top section defining a tear strip in the cap, wherein the tear seams include a sleeve portion extending substantially in parallel and substantially axially from the first and second edges of the pull tab to the top section, and wherein the tear seams have a top portion continuous with the sleeve portion and partially circumscribing the top section;
wherein the sleeve section includes a segmented snap ring protruding radially inward on the inner surface of the sleeve section except at a pair of gaps coinciding with the pair of tear seams; and
wherein the top section includes a seal cylinder projecting concentrically from the top section to seal against an inside surface of the cannula connector end.

2. The cap of claim 1 wherein the tears seams have a U-shaped profile.

3. The cap of claim 1 wherein the snap ring is configured to mate with a barb on the cannula.

4. The cap of claim 1 wherein the seal cylinder projects as a hollow cylindrical rib to engage the cannula connector end.

5. The cap of claim 4 wherein the top section includes a plurality of vent holes disposed radially inward of the cylindrical rib.

6. The cap of claim 1 wherein the pull tab includes a grasping pad that widens out from the first and second edges.

7. The cap of claim 1 molded as a single integrated unit.

8. The cap of claim 1 comprised of polyethylene.
